(19) United States
(12) Patent Application Publication
(10) Pub. No.: US 2009/0118624 A1
(43) Pub. Date: May 7, 2009

(54) DEVICE FOR ORAL CAVITY EXAMINATION

(75) Inventor: Mark BRIDE, Preoria, AZ (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: ZILA PHARMACEUTICALS, INC., Scottsdale, AZ (US)

(21) Appl. No.: 12/351,632

(22) Filed: Jan. 9, 2009

Related U.S. Application Data
(63) Continuation-in-part of application No. 12/101,023, filed on Apr. 10, 2008.
Application No. 12/101,023 is a continuation-in-part of application No. 11/400,110, filed on Apr. 6, 2006, now abandoned.
Application No. 11/400,110 is a continuation-in-part of application No. 10/564,800, filed on Jan. 12, 2006, now abandoned.

(51) Int. Cl. A61B 6/00

(52) U.S. Cl. 600/476

(57) ABSTRACT

Described herein is a device used to perform examinations for detection of abnormal epithelial tissue, the device includes a handle portion, a housing and a mirror assembly secured to an end of the housing. The housing has first and second ends and is detachably connectable to the handle. The housing defines an interior and an opening that communicates the interior with an exterior of the housing when the handle and housing are connected.

### RELATED APPLICATIONS

This application is a continuation-in-part of application Ser. No. 12/101,023, filed Apr. 10, 2008, which is a continuation-in-part of application Ser. No. 11/400,110, filed Apr. 6, 2006, which is a continuation-in-part of application Ser. No. 10/564,800, filed Sep. 28, 2004, the contents of each of which are incorporated by reference in their entireties.

### FIELD OF THE INVENTION

The present invention relates generally to devices for detecting abnormal epithelial tissue that may harbor tumor phenotypes.

### BACKGROUND OF THE INVENTION

Patients who delay in obtaining a cancer consultation may have significantly higher relative hazards of death than do patients with a shorter delay. Thus, if patients are more regularly subjected to effective cancer screening, the mortality risks of cancer could be reduced. There is a need for a simple, rapid screening test for the detecting abnormal mucosal tissue which may harbor tumor phenotypes, which may indicate the presence of or the eventual development of invasive cancer.

Abnormal epithelial tissue can be visually identified and located real time in vivo using selective light examination, which are admirably suited for rapid and inexpensive screening carried out as routine medical and dental examinations. Illustratively, U.S. Pat. Nos. 5,179,938 and 5,329,938 to Lonky, and U.S. Patent Application Publication Nos. 2006/0241494 and 2006/0241501 to Bride, the contents of each of which are incorporated herein by reference in their entireties, describe instruments equipped with a chemiluminescent light source which radiates in the visible green, blue, and optionally, red spectrums, with spectral peaks at 430, 550, and 580 nm. Under such illumination, with normal ambient light surpressed, abnormal mucosal tissue appears white. Illustratively, such selective light devices for practicing such in vivo examinations are commercially available under the registered trademark VIZILITE® from Zila Pharmaceuticals, Inc., Phoenix, Ariz., USA.

### SUMMARY OF THE PREFERRED EMBODIMENTS

According to a first aspect of the present invention, there is provided a device used to perform examinations for detection of abnormal epithelial tissue. The device includes a handle portion, a housing and a mirror assembly secured to an end of the housing. The housing has first and second ends and is detachably connectable to the handle. The housing defines an interior and an opening that communicates the interior with an exterior of the housing when the handle and housing are connected. In a preferred embodiment, the sleeve includes an opening defined therein that is aligned with the opening in the housing for emitting light therethrough.

According to another aspect of the present invention, there is provided a method of detecting abnormal epithelial tissue. The method includes the steps of providing a light source and a housing, inserting the light source into the housing, illuminating an area of epithelial tissue with incident light emitted from the light source, and viewing reflected light in a mirror. The incident light is directed through the opening in the housing, and at least a portion of the incident light is reflected from the area, which creates the reflected light having at least one wavelength. The mirror is secured to the housing.

According to another aspect of the present invention, there is provided a method that includes providing a device used to perform examinations for detection of abnormal epithelial tissue. The device includes a handle portion and a housing having first and second ends. The housing defines an interior and an opening that communicates the interior with an exterior of the housing when the handle and housing are connected. The method also includes inserting a light source through an opening in the first end of the housing, inserting a male connection member on the handle portion into the opening in the first end of the housing, and sliding a mirror assembly onto the second of the housing. In a preferred embodiment, the mirror assembly includes a sleeve that is slid onto the second end of the housing.

According to another aspect of the present invention there is provided a kit for medical examination. The kit includes a device that has a handle portion and a housing that includes first and second ends that and is detachably connectable to the handle. The housing defines an interior and an opening that communicates the interior with an exterior of the housing when the handle and housing are connected. The kit also includes a mirror assembly, and a light source.

### BRIEF DESCRIPTION OF THE DRAWINGS

The invention may be more readily understood by referring to the accompanying drawings in which:

FIG. 1 is a graph showing the preferred spectral output in accordance with an embodiment of the present invention;

FIG. 2 is a graph showing preferred lens transmission in accordance with an embodiment of the present invention;

FIG. 3 is an exploded perspective view of a retractor and chemiluminescent light source in accordance with an embodiment of the present invention;

FIG. 4 is a perspective view of the assembled retractor of FIG. 3;

FIG. 5 is a perspective view of an examination of the oral cavity using the retractor of FIG. 3;

FIG. 6 is a cross-sectional view of the retractor of FIG. 3 taken along line 6-6 of FIG. 4;

FIG. 7 is a perspective view of a device used for medical examination in accordance with a preferred embodiment of the present invention;

FIG. 8 is a cross-section of the device of FIG. 7 taken along line 8-8 of FIG. 7;

FIG. 9A is a perspective view of the device of FIG. 7;

FIG. 9B is a cross-sectional side view of the device of FIG. 7 taken along line 9B/9C-9B/9C of FIG. 9A and showing a hollow handle;

FIG. 9C is a cross-sectional side view of the device of FIG. 7 taken along line 9B/9C-9B/9C of FIG. 9A and showing a solid handle;

FIG. 10 is a perspective view of a device used for medical examination in accordance with another embodiment of the present invention with a portion of a sidewall of the device removed;

FIG. 11 is a cross-section of the device of FIG. 10; taken along line 11-11 of FIG. 10;

FIG. 12A is a cross-sectional side view of the device of FIG. 11 taken along line 12A/12B-12A/12B of FIG. 10 and showing a hollow handle;

FIG. 12B is a cross-sectional side view of the device of FIG. 11 taken along line 12A/12B-12A/12B of FIG. 10 and showing a hollow handle;

FIG. 13 is a perspective view of a device used for medical examination in accordance with another embodiment of the present invention;

FIG. 14A is a cross-sectional side view of the device of FIG. 13 taken along line 14A/14B-14A/14B of FIG. 13 and showing a hollow handle;

FIG. 14B is a cross-sectional side view of the device of FIG. 13 taken along line 14A/14B-14A/14B of FIG. 13 and showing a hollow handle;

FIG. 15 is a diagram of the light emitted from a light source including reflected light;

FIG. 16 is a perspective view of the retractor and chemiluminescent light source of FIG. 3 including an attachable mirror assembly;

FIG. 17 is a side elevational view of the mirror assembly of FIG. 16;

FIG. 18 is a top plan view of the mirror assembly of FIG. 16;

FIG. 19 is a cross-section of the mirror assembly of FIG. 16 taken along line 19-19 of FIG. 18;

FIG. 20 is an right side elevational view of the mirror assembly of FIG. 16;

FIG. 21 is a cross-sectional elevational view of the mirror assembly of FIG. 16 on a retractor;

FIG. 22 is a perspective view of an examination of the oral cavity using the retractor of FIG. 3 and the mirror assembly of FIG. 16; and

FIG. 23 is a perspective view of the mirror assembly of FIG. 16 on the device for medical examination of FIG. 7.

Like numerals refer to like parts throughout the several views of the drawings.

### DETAILED DESCRIPTION OF THE PREFERRED EMBODIMENTS

As shown in the drawings, for purposes of illustration, the invention is embodied in an apparatus and method for medical examination (as used herein, may refer to “dental examination”). Preferably, the present invention is used to detect abnormal epithelial tissue (as used herein, tissue characterized as “abnormal” may include any/all of the following: pre-cancerous tissue, cancerous tissue; tissue experiencing angiogenesis; tissue exhibiting molecular/genetic signs of precancer/cancer; tissue having cells with abnormal apoptotic pathways, etc.). The apparatus preferably includes a light source (as used herein, “light source” may refer to any/all of the components of a chemiluminescence reaction). The light source preferably emits light. Preferably, the device includes a reflective layer (as used herein, may refer to “reflective material”) disposed thereon.

Briefly, one embodiment of the invention for screening epithelial tissue for possible abnormal tissue includes illuminating a gross anatomical area of epithelial tissue with the light emitted from the light source. Preferably, at least a portion of the light emitted from the light source is reflected off the reflective layer back into the chemical housing. Additionally, at least a portion of the light emitted from the light source is incident light (as used herein, the incident light and light reflected off the reflective layer back into the chemical housing are collectively referred to as “emitted light”). However, the device is not limited to the detection of abnormal epithelial tissue and may detect other types of tissue/cells.

The emitted light selectively aids in visualizing abnormal tissue sites on the gross area. Abnormal tissue sites preferably appear white or substantially white. A filter lens may be used to view the illuminated gross area of tissue. Use of the filter lens allows the examiner to view reflected light only in certain desired wavelengths, while substantially blocking transmission of ambient or interfering light of wavelengths other than the reflected light of the desired wavelengths, thus enhancing the selective visualization of any abnormal tissue sites in the presence of normal ambient light.

For exemplary purposes only, described hereinbelow is a preferred embodiment wherein the device houses a chemiluminescent light source, 9,10 diphenylanthracene (“DPHA”). DPHA is used as a fluorescer in a peroxyoxalate chemiluminescence system (hydrogen peroxide is used as the energy source). However, this is not a limitation on the present invention. It will be understood that the method and device may use any other type of chemiluminescent light source, or any other light source, for example, incandescent, fluorescent, or the like. Any light source that emits incident light that can be reflected back in a selected spectral range is within the scope of the present invention. Other light sources will be readily apparent to those skilled in the relevant art.

It will be appreciated that terms such as “front,” “back,” “top,” “bottom,” “side,” and the like used herein are merely for ease of description and refer to the orientation of the components as shown in the figures. It should be understood that any orientation of the device, and the components thereof, described herein, is within the scope of the present invention.

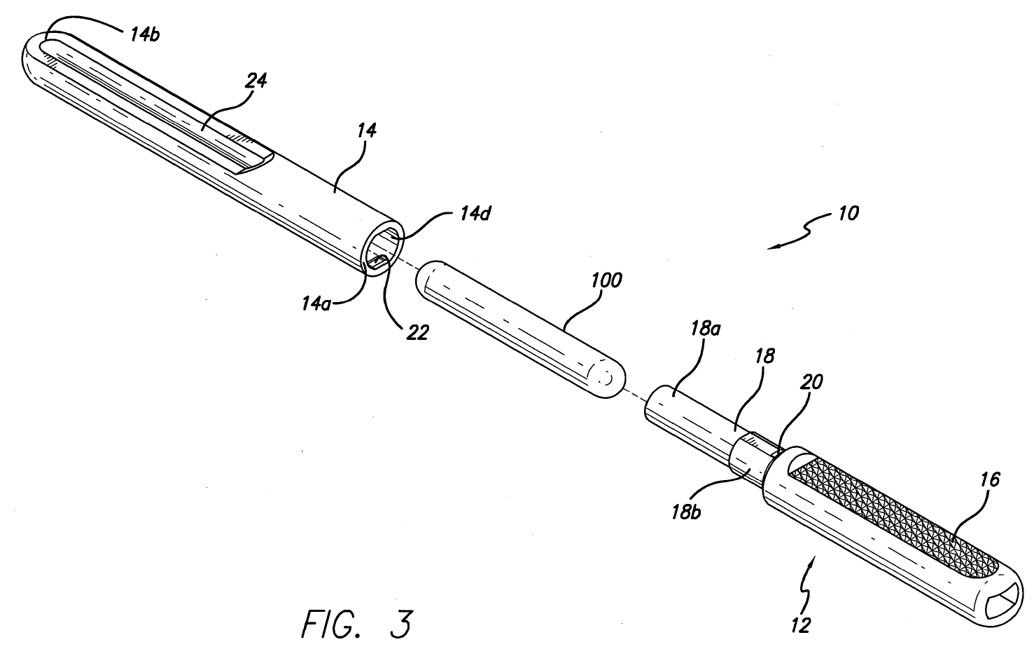

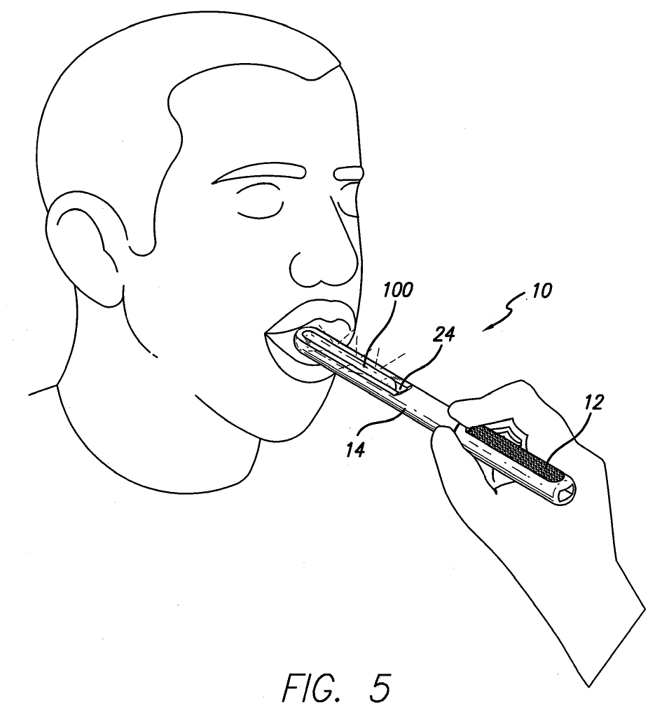

Referring to FIGS. 3-6, a retractor 10 for housing a light source 100 is shown. The retractor 10 includes a handle portion 12 and a sleeve or housing 14 that are detachably connectable. The handle portion 12 includes a handle 16 and a male connection member 18. The housing 14 includes first and second ends 14a and 14b and defines an interior 14c. The first end 14a of the housing defines an opening 14d into which the male connection member 18 can be inserted. In a preferred embodiment, the male connection member 18 includes a protrusive portion 18a and a stepped portion 18b that is sized to be received through the opening 14d in housing 14.

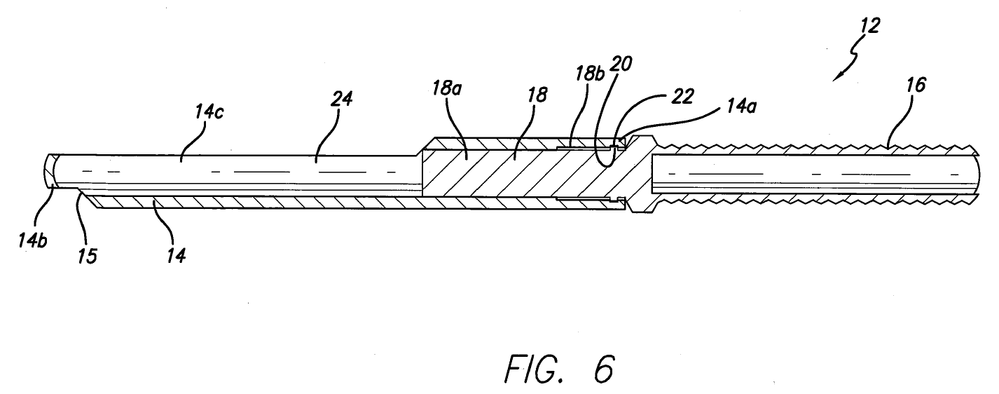

Preferably, the stepped portion 18b includes at least one tab 20 (two are shown in FIG. 6) that is received in a corresponding recess 22 defined in the inside surface of housing 14. This arrangement keeps housing 14 attached to or snap fit on the handle portion 12. To detach the two components a user simply pulls the housing 14 and handle portion 12 in opposite directions. This will disengage the tabs 20 and recesses 22 and pull the housing 14 off of the male connection member 18. In use, because the housing 14 is detachable from the handle portion 12, the housing 14 can be disposed after use.

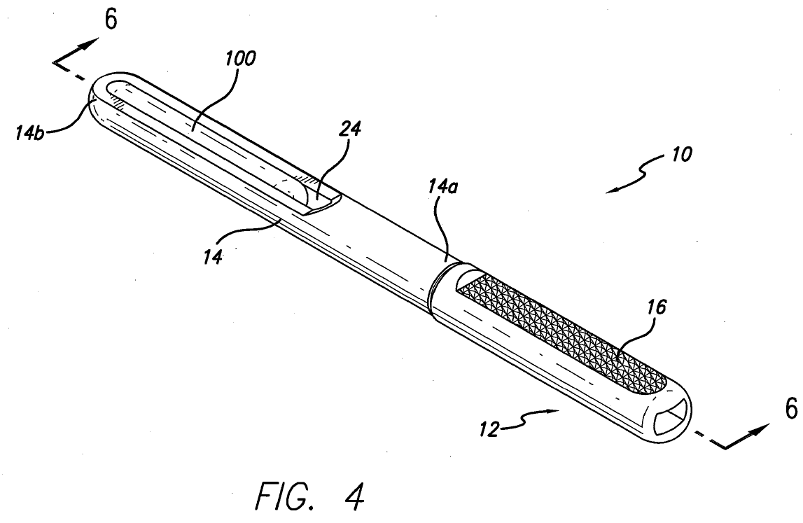

As shown in FIG. 4, the housing 14 is sized to receive a light source 100. The light source 100 is inserted into the housing interior 14c through opening 14d and then is followed by inserting the male connection member 18 through opening 14d to connect the handle portion 12 with the housing 14.

As can be seen in FIG. 3, the housing 14 has an opening 24 defined therein. In a preferred embodiment, the opening 24 is elongated as shown. It will be understood that the housing 14 is made of an opaque material. In a preferred embodiment, the housing 14 is made of a lightweight plastic, such as polystyrene. However, it will be understood that the housing 14 can be made of any material that is opaque and can be formed to house a light source. In another embodiment, the retractor 10 can be made of a transparent material.

FIG. 6 shows another opening on the side of the housing 14 opposite the opening 24 and near the second end 14b. However, this opening can be omitted.

The shape and location of the opening 24 is not a limitation on the present invention. For example, the opening can be smaller or located at the tip of the housing. Any shape and location of the opening that selectively prevents interfering light from being directed toward the examiner's eyes is within the scope of the present invention.

It will be appreciated by those skilled in the art that the spirit of the present invention is to provide a housing or sleeve that surrounds a light source and includes an opening therein for directing incident light toward the tissue to be examined. Any apparatus that directs incident light as described is within the scope of the present invention. For example, U.S. Pat. No. 6,496,718 to Lonky, incorporated herein by reference, teaches a body cavity light that uses an omni-directional diffuse light source. It is within the scope of the invention to provide a sleeve or housing that covers the diffuse light source and only allows the incident light from the diffuse light source to emanate from an opening in the sleeve or housing (thereby making it non-omni-directional). In other embodiments, the housing can be flexible or extendable. For example, in an embodiment where the housing is flexible and/or extendable a mechanism, such as a fiber through which light is transmitted (e.g., fiberoptics), for transmitting the light from the light source to the opening in the housing would preferably be included. The housing can also be disposable or sterilizable.

In another embodiment, the light source can be connected to the handle and the housing or sleeve can be removable from the handle and housing combination.

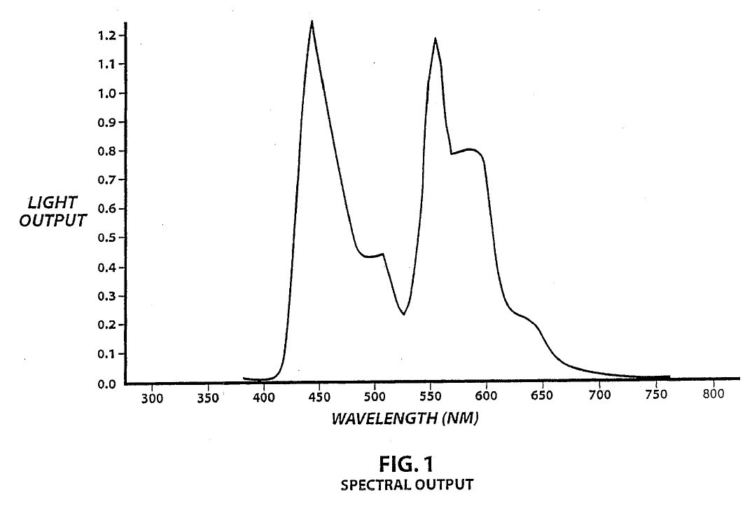

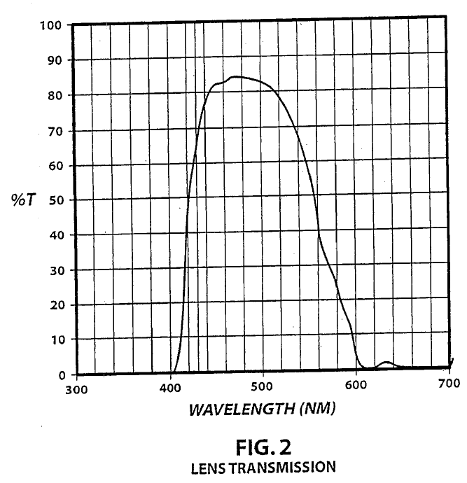

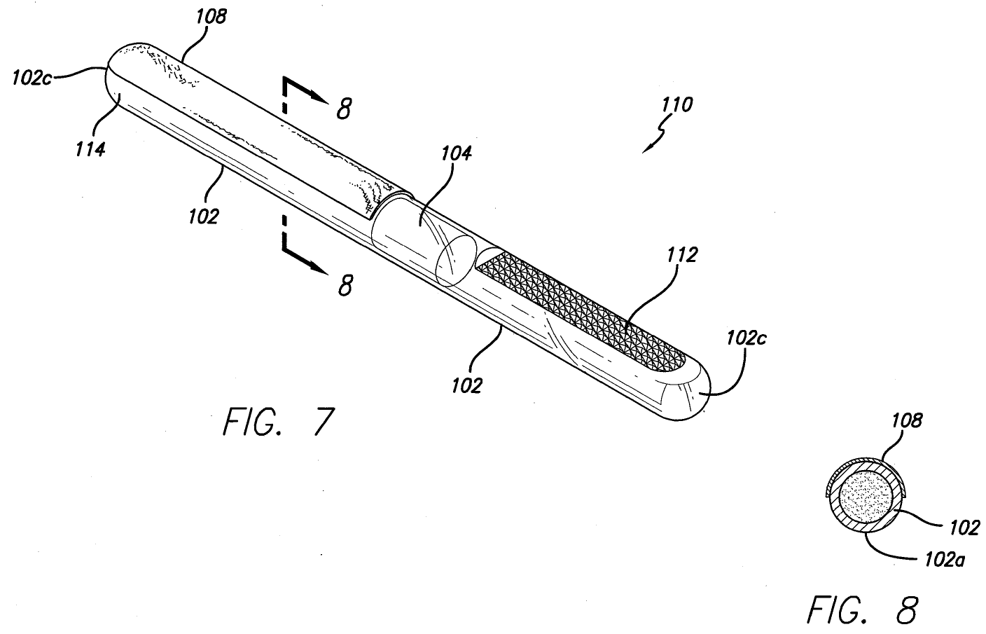

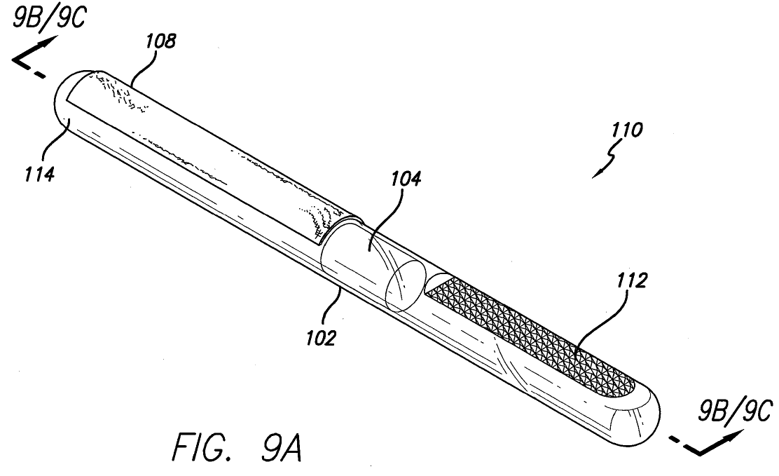

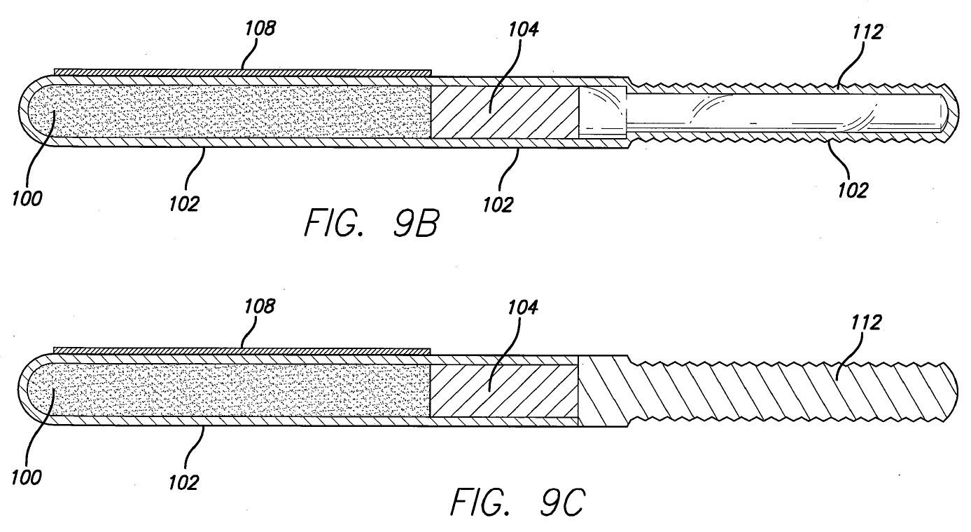

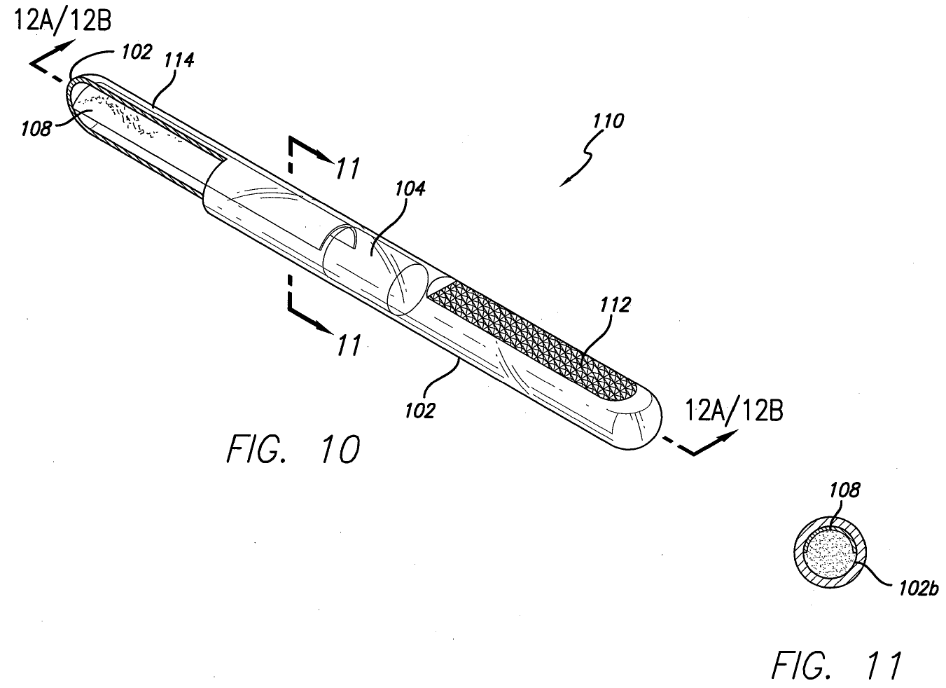

Referring to FIGS. 7-15, a device 110 used for medical examination is described. In a preferred embodiment, the device 110 is a self-contained, one-piece device. Preferably, the device 110 includes a sidewall 102, a chemical housing 114, a handle portion 112, and two ends 102c (referred to individually and collectively as “102c”). Accordingly, and as seen in FIGS. 8-12B, the device 110 is preferably tubular in shape. The sidewall 102 preferably defines both the entire chemical housing 114 and the entire handle portion 112. As such, the chemical housing 114 is preferably contiguous with the handle portion 112. As best seen in FIGS. 1 and 2, a reflective material or layer 108 is preferably applied to an outer surface 102a of at least a portion of the sidewall 102 defining the chemical housing 114. Preferably, the reflective material or layer 108 is a reflective tape. As best seen in FIGS. 9B-9C, the chemical housing 114 preferably includes a light source 100 disposed therein.

In other embodiments, the sidewall 102 may define the entire chemical housing 114 and a portion of the handle portion 112, or may define a portion of the chemical housing 114 and the entire handle portion 112. Likewise, in other embodiments, the sidewall 102 may define a portion of the chemical housing 114 and a portion of the handle portion 112. Accordingly, a portion of the chemical housing 114 and/or a portion of the handle portion 112 may be defined by another sidewall or other suitable component, without departing from the scope of the present invention.

The reflective layer 108 is preferably applied as follows. Referring now to FIGS. 7 and 8, the reflective layer 108 is preferably applied along the length of the sidewall 102 defining the chemical housing 114. Since the device 110 is preferably tubular in shape, a cross-section of the device 110 is circular or substantially circular and is therefore 360°. In a preferred embodiment, the reflective layer 108 is applied around about 50° to about 300° of the cross-section. In a more preferred embodiment, the reflective layer 108 is applied around about 100° to about 250° of the cross-section. In a highly preferred embodiment, the reflective layer 108 is applied around about 180° of the cross-section. Accordingly, the reflective layer 108 is preferably applied on about 50% of the circumference of the outer surface 102a of the sidewall 102. In other embodiments, the reflective layer 108 may be applied less than about 180° or more than 180° around the outer surface 102a of the sidewall 102 defining the chemical housing 114. Additionally, it is to be understood that the reflective layer 108 may be applied along the length as well as the width of the sidewall, or applied to the width alone, without departing from the scope of the present invention. It should be applied sufficiently to the sidewall 102 to provide the intended reflective effect.

As best seen in FIGS. 10-12B, in other embodiments, the reflective layer 108 may be applied to an inner surface 102b of the sidewall 102 defining the chemical housing 114. As such, the reflective layer 108 is disposed on an inside of the chemical housing 114. A reflective layer 108 applied to the inner surface 102b operates similarly to a reflective layer 108 applied to the outer surface 102a: at least a portion of the light emitted from the light source 100 reflects off the reflective layer 108 back into the chemical housing 114 and at least a portion of the light emitted from the light source 100 is incident light. Accordingly, it is to be understood that the discussion that pertains to the reflective layer 108 applied to the outer surface 102a of the sidewall 102 also pertains to the embodiments wherein the reflective layer 108 is applied to the inner surface 102b.

Additionally, it is to be understood that the reflective material or layer 108 may be applied (as used herein, interchangeable with “disposed” or “printed”) to the sidewall 102 in any fashion and/or any configuration. Accordingly, the reflective material or layer 108 may include an adhesive on one or both of its sides, and/or or the sidewall 102 may include an adhesive on its outer and/or inner surfaces for attaching the reflective layer 108 to the sidewall 102. Additionally, more than one reflective layer 108 may be applied to the sidewall 102. When more than one reflective layer is used, the reflective layers may at least be partially disposed on top of each other, or may be applied in separate locations on at least a portion of the sidewall 102 defining the chemical housing 114. In other embodiments, the reflective layer 108 may be applied to at least a portion of the sidewall 102 defining the chemical housing 114 and at least a portion of the sidewall 102 defining the handle portion 112. Additionally, the reflective material 108 may be spotted onto the sidewall 102.

Preferably, the reflective layer 108 is comprised of plastic. Examples of plastics include, but are not limited to, polyethylene, polyvinyl chloride, polyvinylidene chloride, polypropylene, polyamide, polycarbonate, polytetrafluoroethylene, polyurethane, polystyrene, or the like. It is to be understood that the reflective layer 108 may be made out of any suitable material that has a high reflective index. Examples of such materials include polystyrene, styrene-methyl methacrylate copolymers, (meth)acrylic resin, polymethyl pentene, allyl glycol carbonate resin, spirane resin, amorphous polyolefin, polycarbonate, polyamide, polyallylate, polysulfone, polyallyl sulfone, polyether sulfone, polyether imide, polyimide diallyl phthalate, fluoro-resins, polyester carbonate, resin of norbornene family (ARTON), alicylic acrylic resin, silicone resin, acrylic rubber, and silicone rubber.

In a preferred embodiment, the thickness of the reflective layer 108 is from about 2 mm to about 0.02 mm. In a more preferred embodiment, the thickness of the reflective layer 108 is from about 11.0 mm to about 0.04 mm. In a highly preferred embodiment, the thickness of the reflective layer 108 is from about 0.07 mm to about 0.05 mm. However, the thickness of the layer may be more than 2 mm or less than 0.04 mm without departing from the scope of the present invention. Generally, when the reflective layer 108 is too thin, the efficiency of light reflection is reduced. Accordingly, the thickness of the reflective layer 108 should take this into account. As such, the reflective layer 108 is preferably of a thickness that maximizes the efficiency of light reflection and/or maximizes light output.

In a preferred embodiment, the sidewall 102 defining the chemical housing 114 is translucent. This allows the emitted light to be transmitted to the outside. In other embodiments, the housing or portions thereof may be opaque or substantially opaque. In other embodiments, any suitable material that allows the light source 100 to transmit light through at least a portion of the sidewall 102 defining the chemical housing 114 is within the scope of the present invention. For example, at least a portion of the sidewall 102 may be transparent or translucent. In some embodiments, the material of the sidewall 102 defining the chemical housing 114 may be chosen so as to maximize light output.

Referring now to FIGS. 7-15, the device 110 preferably operates as follows. Preferably the chemical housing 114 is bent in order to activate the chemiluminescence reaction. Bending may mix the components of the chemiluminescence reaction in the following manner: The chemical housing 114 may be comprised of two compartments. A first (inner) compartment is breakable, such as a frangible ampoule, and contains one of the components, either the activator of the fluorescer. A second (outer) compartment (preferably defined by the sidewall 102) is partially or fully flexible, but is sturdy enough to resist being cut or broken when the ampoule is broken. It contains the other component. Accordingly, bending the sidewall 102 defining the chemical housing 114 at least partially breaks the inner compartment, exposing the contents therein to the contents of the outer compartment in the chemical housing 114, thereby initiating the chemiluminescence reaction. Accordingly, light is emitted from the reaction. In other embodiments, the light source 100 may be activated by other means. For example, a plunger may be used to mix one component with another in the chemical housing 114.

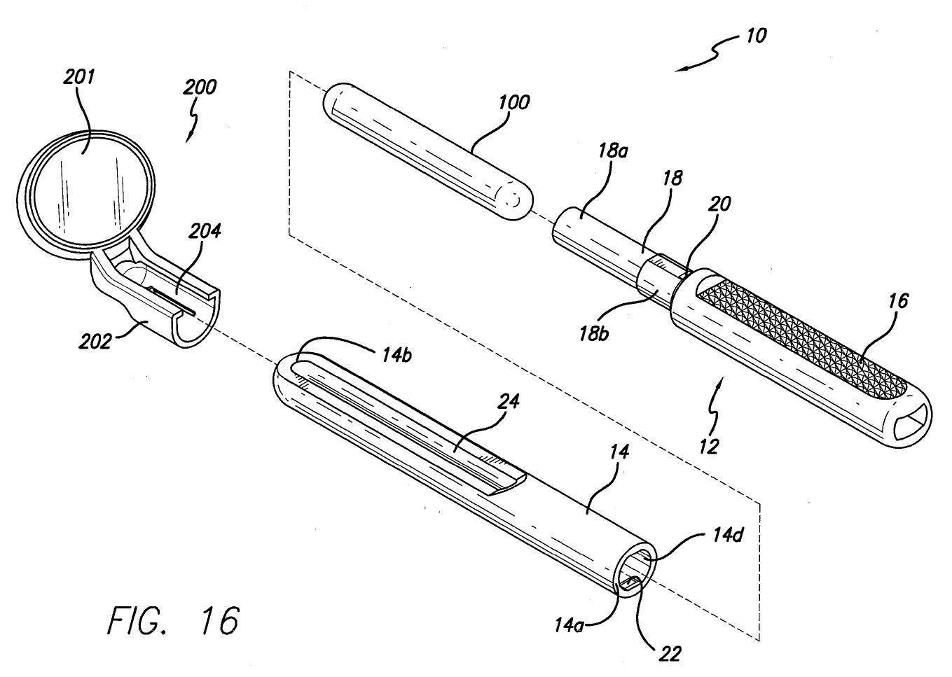

As best seen in FIG. 15, at least a portion of the light emitted from the light source 100 is reflected off the reflective layer 108 back into the chemical housing 114. The light reflected off the reflective layer 108 is represented by “310.” Additionally, a portion of the light emitted from the light source is incident light, which is represented by “320.” In a preferred embodiment, the light reflected off the reflective layer 310 and the incident light 320 together provide a light output that is greater than the light output of the incident light 320 alone. An additive effect of the light 310 reflected off the reflective layer 108 and the incident light 320 preferably provides this increase in light output.

Preferably, the light source 100 of the device 110 is directed to either the oral cavity or the vaginal cavity and preferably to the tissue/cells of interest. As such, the portion of the sidewall 102 not covered by the reflective layer 108 preferably faces the tissue of interest. The emitted light is thus directed to the tissue/cells of interest. Abnormal tissue generally appears different than normal tissue. Abnormal tissue appears white or substantially white, and regular tissue does not appear white. The tissue/cells of interest may be viewed in the dark or in ambient light preferably through a filter lens.

The size of the device 110 may vary depending upon its intended use. In a preferred embodiment, the size of the chemical housing 114 is large enough to contain a sufficient amount of chemiluminescent material to light at least a portion of the cavity to be examined. The device should be of a size that is capable of being disposed within the cavity adjacent the area of the cavity to be examined. Accordingly, a device suitable for vaginal use may be smaller than a device suitable for oral use.

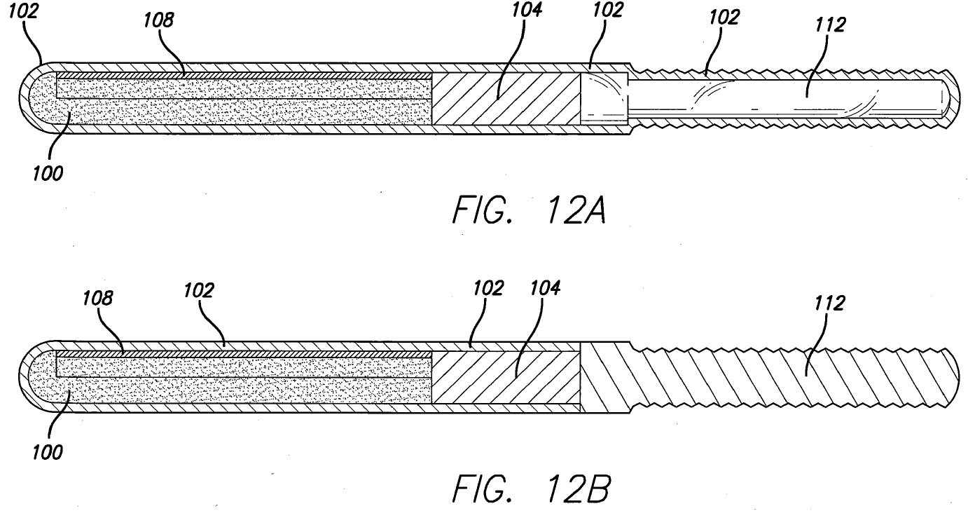

In a preferred embodiment, and as seen in FIGS. 9B and 12A, the handle portion 112 may be hollow. However, the handle portion 112 may not have an inside, as seen in FIGS. 9C and 12B. Additionally, in a preferred embodiment, the sidewall 102 defining the handle portion 112 is opaque or substantially opaque. In other embodiments, the handle portion 112 may be made of any suitable material. The material may or may not be able to transmit light.

Additionally, in some embodiments, a stop deposit 104 may be disposed between the light source 100 and the handle portion 112. The stop deposit 104 is preferably defined by the sidewall 102. It is preferably used to separate the light source from the handle portion 112. The stop deposit 104 may be made out of wax or paraffin; however, any material that does not absorb, or only partially absorbs, the light source 100 (i.e., the chemicals within the light source) is within the scope of the present invention. In other embodiments, the stop deposit 104 may be omitted or may be replaced by another suitable structure.

Additionally, it is to be understood that the device of the present invention may be stored for long periods of time without loss of function, as the light source is not activated unless the chemical components are mixed together.

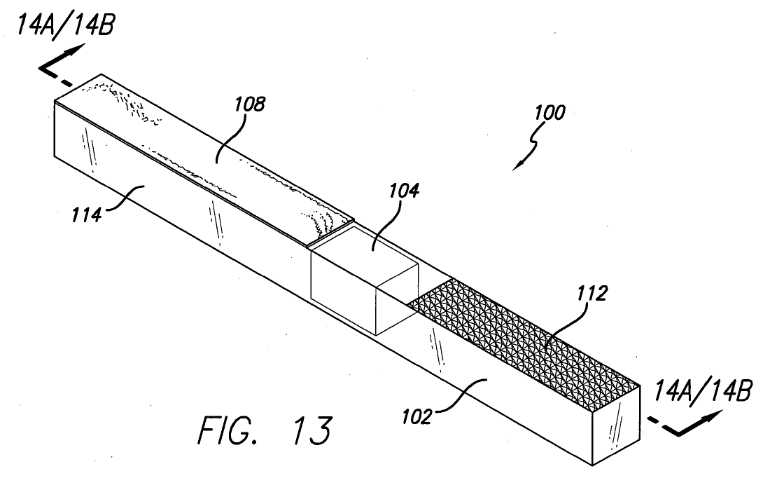

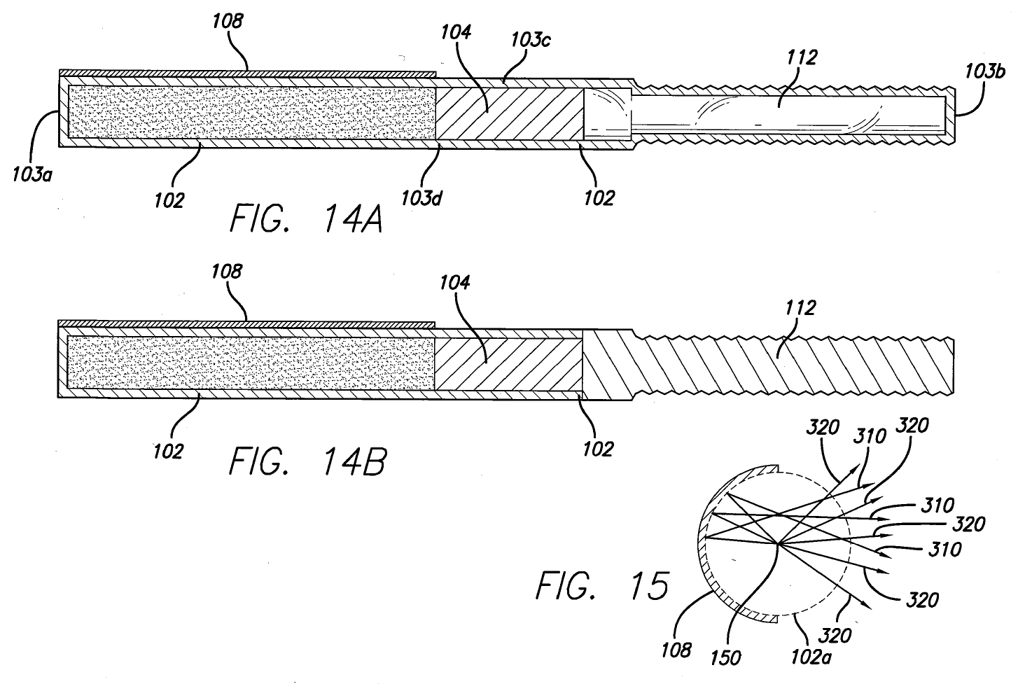

Referring now to FIGS. 13-14B, in other embodiments, the device 110 may be rectangular in shape. A device 110 that is rectangular in shape generally has four sidewalls (as seen in FIG. 14A, represented individually by 103a-d and collectively by 102). In a preferred embodiment, the reflective layer 108 is applied from about 10% to about 85% of the outer surface of the sidewall 102. In a more preferred embodiment, the reflective layer 108 is applied from about 25% to about 70% of the outer surface of the sidewall 102. In a highly preferred embodiment, the reflective layer 108 is applied to about 50% of the outer surface of the sidewall 102. In other embodiments, the reflective layer 108 may be applied to more than 50% or less than 50% of the outer surface of the sidewall 102.

In another embodiment of the present invention, there is provided a kit for medical examination. The kit preferably includes the device of the present invention as well as a light source contained therein. The device preferably includes a reflective tape applied thereon. Spectacles having a filter are preferably included in the kit.

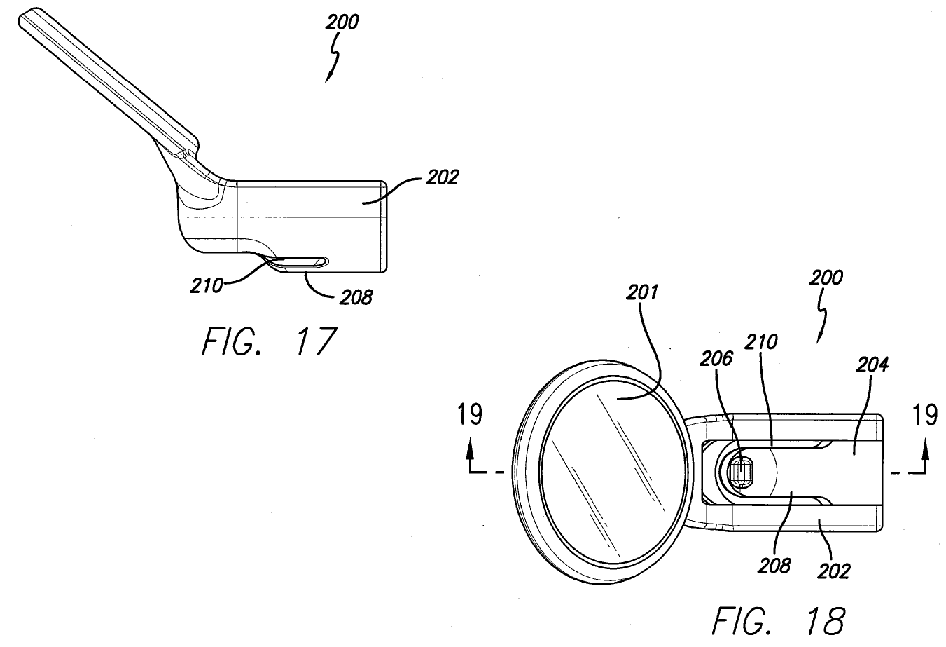

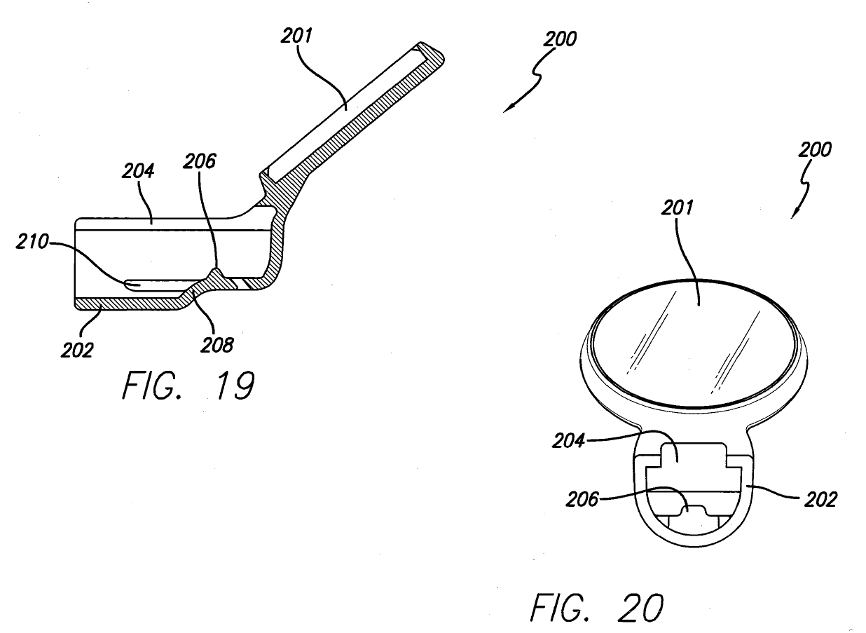

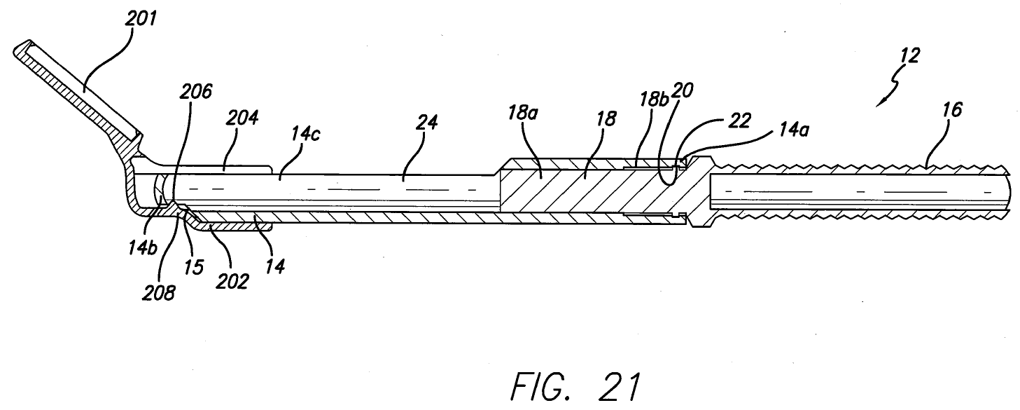

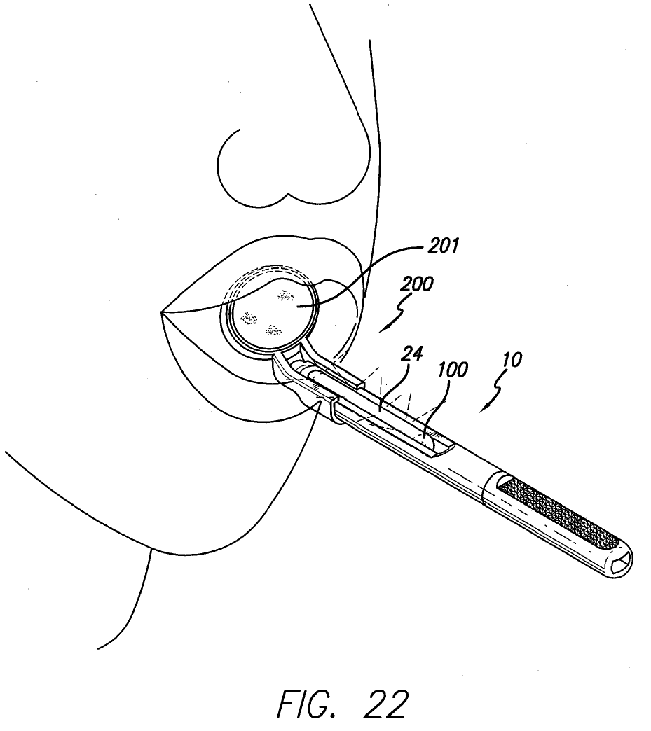

As shown in FIGS. 16-21, in another embodiment, the device or retractor 10 can include a mirror assembly 200 having a mirror 201 for aiding during an examination. The tonsillar crypts and base of the tongue are particularly difficult to inspect during the course of oral examination by direct visualization. As shown in FIG. 21, the mirror assembly 200 can be attached to the end 14b of housing 14 and opposite the handle 16. However, the mirror assembly 200 can be attached to other areas of the retractor 10 as well.

Any method or procedure for attaching the mirror to the housing is within the scope of the present invention. In an exemplary embodiment, the mirror assembly 200 includes a sleeve 202 having a recess 204 defined therein. As shown in FIG. 21, the sleeve 202 receives the end 14b of housing 14 and the recess 204 aligns with opening 24 to allow light emitted to pass therethrough.

As shown in FIGS. 6 and 21, the housing 14 includes an opening 15 therein near second end 14b. The sleeve 202 includes a spring biased knob 206. As shown in FIGS. 17-21, the knob 206 is located near the end of an arm 208 that has been formed in the sleeve 202 by defining a space 210. In a preferred embodiment, because sleeve 202 is made of, for example, glass bead filled polypropylene, when the housing 14 is received in sleeve 202, the knob 206 will bend backwards as it rides along the bottom of the housing 14 until it is received in opening 15, thereby securing the mirror assembly 200 on the end of the housing 14. In a preferred embodiment, the sleeve 202 and the housing 14 are aligned axially. In alternative embodiments, the sleeve 202 and its components can be made of other plastics, such as polyimide thermoplastic resin, polyphenylsulphone or polysulphone.

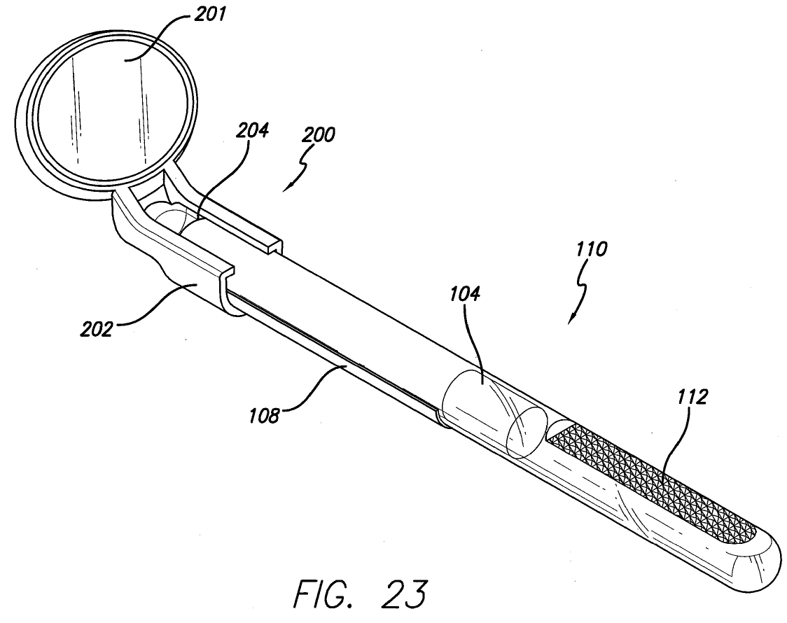

FIG. 23 shows the mirror assembly 200 on device 110 described above.

In other embodiments, the mirror 201 can be secured by threading it onto the housing 14, it can be permanently attached to the housing, it can be formed unitarily with the housing. It will be understood that the method for associating the mirror with the housing is not a limitation on the present invention.

The following examples are presented to enable those skilled in the art to understand and practice the invention and to identify the presently preferred embodiments thereof. These examples are provided for illustrative purposes and not to indicate the scope of the invention which is defined only by the appended claims.

### EXAMPLE 1

A routine visual examination of the oral cavity is made, noting the presence or absence of any lesions on the attached gingival, the buccal mucosa, the floor of the mouth, the hard and soft palate, and the dorsal, lateral, and ventral tongue. The presence or absence of any lesions noted by this routine examination are recorded. Additionally, the presence or absence of clusters of blood vessels (i.e., angiogenesis) which may indicate new growth such as cancer is noted.

### EXAMPLE 2

After completing the routine examination of Example 1, the patient is then instructed to rinse the mouth with a 1% acetic acid solution for up to one minute and then expectorate.

Referring to FIG. 1, the device 110 of the present invention is activated by bending the sidewall 102 defining the chemical housing 114, thereby allowing the components therein to mix together.

Preferably, and as indicated earlier, 9,10 diphenylanthracine (“DPHA”) is used as the light source, and the light provided has a spectral peak of about 430 nm. This spectral peak preferably produces a blue light. In a preferred embodiment, use of DPHA reduces the amount of mucosal glare and provides a softer light than the use of other chemiluminescent agents. In other embodiments, the chemiluminescent light source described in U.S. Pat. No. 5,329,938 to Lonky, the entire contents of which are herein incorporated by reference, may be used. The light source described in that patent is commercially available under VIZILITE. The light provided has spectral peaks at about 430 nm, 550 nm, and a smaller peak in the red region at about 600 nm, and the spectral peaks produce a bluish-white light.

In a preferred embodiment, the examining clinician then dons a pair of spectacles provided with at least one lens which only transmits light in the wavelength band of 400-600 nm. These spectacles are shaped to minimize illumination reaching the examiner's eyes from above and from the sides. For example, the spectacles as disclosed in U.S. Patent Application Publication No. 2006/0241494 to Bride, the contents of which are herein incorporated by reference in their entirety, may be used.

Without reducing ambient light from normal illumination sources, the visual examination of the oral cavity is then repeated using the illumination provided by the light source, looking for lesions or other suspect tissue sites that appear white, paying attention to any suspect tissue sites noted in the routine examination of Example 1. Any sites which appear white or bluish white are noted and recorded.

Further assessment of the noted sites is made, for example by tissue biopsy for standard histology or by molecular analysis, to determine whether the tissue is cancerous or harbors mutations which are in the pathway for eventual development of invasive cancer. Molecular analysis may include LOH (Loss of Heterozygosity), such as microsatellite analysis, or the like.

### EXAMPLE 3

After completing the routine examination of Example 1, the patient is then instructed to rinse the mouth with a 1% acetic acid solution for up to a minute and then expectorate. Referring to FIGS. 1 and 2, the light source 100 of the device 110 of the present invention is then activated by bending the sidewall 102 defining the chemical housing 114.

The light source 100 emits light when activated. A portion of this light reflects off the reflective layer and travels back into the chemical housing. Preferably, at least a portion of this light is transmitted through the sidewall 102 defining the chemical housing 114. Additionally, a portion of the light emitted is incident light (as indicated earlier, collectively referred to herein as “emitted light”). Preferably, the light provided has a spectral peak at about 430 nm.

The emitted light is directed to the suspected pre-cancerous or cancerous region. The device is preferably manipulated so that little or none of the emitted light is directed toward the eyes of the examiner before being reflected. The examination is performed looking for lesions or other suspect tissue sites which appear white, paying attention to any suspect tissue sites noted in the routine examination of Example 1. Any sites which appear white or bluish-white (or “purplish” (due to red lesions absorbing the blue wavelength of light)) are noted and recorded.

For any portions of the mouth that are difficult to view, especially the structures of the throat, the mirror assembly 202 can be placed on the housing 14 or 114. The emitted light is directed to the suspected pre-cancerous or cancerous region and the device is manipulated so that the user can see the suspect region in the mirror 201. It will be understood that the mirror 201 is not used to direct the light to the suspect region. The mirror 201 is used to view the light reflected by the suspect region.

### EXAMPLE 4

A routine medical examination of the vaginal cavity, such as a routine pelvic exam, is conducted using an endoscope and/or a speculum. For example, a visual examination of the vaginal cavity is conducted, noting the presence or absence of any lesions, irregular vascularature, exophytic regions, ulcerations, and other aptypias of the cervix and vaginal cavity. A speculum may be used to spread apart the vaginal walls, allowing the inside of the vagina and cervix to be examined. The speculum may be plastic, metal, or any other material. The presence or absence of any lesions noted by this routine examination is recorded.

### EXAMPLE 5

The vaginal mucosal membrane is then washed with a solution of 1 to 10% acetic acid. The device of the present invention is then bent in order to mix the components of the light source together. In a preferred embodiment, the light provided has a spectral range of 430 nm to 580 nm with a peak a spectral peak at 430 nm. This spectral peak produces a bluish light.

A speculum or other suitable structure/endoscope is used to hold the vaginal cavity open. The device is then inserted into the vaginal passage so that the light source is directed to the tissue of interest in the vaginal cavity. It is to be understood that the speculum may be disposable or may be sterilized after use.

Further assessment of the noted sites is made, for example by tissue biopsy for standard histology or by molecular analysis, to determine whether the tissue is cancerous or harbors mutations which are in the pathway for eventual development of cancer. Molecular analysis may include PCR and/or microsatellite analysis.

It will be understood that the present invention can be used for examination of other areas of the body, besides the oral or vaginal cavities. For example, the present invention can be used to perform an endoscopic examination of the esophagus, the cervix, or the colon, to detect cancer or the early signs of cancer. In this type of examination, a sleeve or the like with an opening for emitting light is inserted into the patient's esophagus, cervix, or colon and the examination is performed. As will be understood by those skilled in the art of endoscopy, the inside of the organ and the reflected light is viewed remotely on a monitor.

The embodiments described above are exemplary embodiments of the present invention. Those skilled in the art may now make numerous uses of, and departures from, the above-described embodiments without departing from the inventive concepts disclosed herein. Accordingly, the present invention is to be defined solely by the scope of the following claims.

What is claimed is:

1. A method of detecting abnormal epithelial tissue, comprising:
providing a light source,
providing a housing having an opening defined therein, wherein the housing includes a mirror secured thereto,
inserting the light source into the housing,
illuminating an area of epithelial tissue with incident light emitted from the light source, wherein the incident light is directed through the opening in the housing, and wherein at least a portion of the incident light is reflected from the area, thereby creating reflected light having at least one wavelength, and
viewing the reflected light in the mirror.

2. The method of claim 1, wherein the at least one wavelength of the reflected light is between about 400 nm and about 750 nm.

3. The method of claim 1, wherein the illuminating step comprises directing incident light emitted from a chemiluminescent light source towards the area of epithelial tissue.

4. The method of claim 2, further comprising filtering the reflected light to substantially remove wavelengths other than the at least one wavelength, thereby creating filtered light, and viewing the filtered light.

5. The method of claim 4, wherein the step of filtering further comprises substantially removing any ambient light not coming from the light source.

6. The method of claim 4, further comprising providing spectacles having a filter, and wherein the step of filtering the reflected light comprises filtering the reflected light with the spectacles to only allow filtered light through.

7. A device used to perform examinations for detection of abnormal epithelial tissue, the device comprising:
a) a handle portion,
b) a housing having first and second ends that is detachably connectable to the handle, wherein the housing defines an interior and an opening that communicates the interior with an exterior of the housing when the handle and housing are connected, and
c) a mirror assembly affixed to an end of the housing.

8. The device of claim 7 further comprising a chemiluminescent light source disposed in the housing.

9. The device of claim 7 wherein the mirror assembly includes a sleeve that receives an end of the housing.

10. The device 9 wherein the sleeve includes an opening defined therein, wherein the opening in the housing is aligned with the opening in the sleeve for emitting light therethrough.

11. The device of claim 9 wherein the sleeve includes a spring biased knob that is received in an opening defined near the end of housing 14.

12. The device of claim 10 wherein the knob is disposed on the end of an arm that is formed in the sleeve by defining a space therearound.

13. A method comprising the steps of:
a) providing a device used to perform examinations for detection of abnormal epithelial tissue that includes
i) a handle portion,
ii) a housing having first and second ends, wherein the housing defines an interior and an opening that communicates the interior with an exterior of the housing when the handle and housing are connected,
b) inserting a light source through an opening in the first end of the housing,
c) inserting a male connection member on the handle portion into the opening in the first end of the housing, and
d) sliding a mirror assembly onto the second of the housing.

14. The method of claim 13 wherein the mirror assembly includes a sleeve, and wherein the sleeve is slid onto the second end of the housing.

15. The method of claim 14 wherein when the sleeve is slid onto the second end of the housing, a knob on the sleeve is received in an opening defined near the second end of the housing.

16. A kit for medical examination comprising:
(a) a device including:
(i) a handle portion,
(ii) a housing having first and second ends that is detachably connectable to the handle, wherein the housing defines an interior and an opening that communicates the interior with an exterior of the housing when the handle and housing are connected, and
(b) a mirror assembly, and
(c) a light source.

17. The kit of claim 16 wherein the mirror assembly includes a sleeve that is adapted to be received on the second end of the housing.

18. The kit of claim 17 wherein the light source is chemiluminescent.

19. The kit of claim 18, further comprising spectacles having a filter.

20. The device of claim 7, wherein the light source is 9,10 diphenylanthracene.
